(19) United States
(12) Patent Application Publication
(10) Pub. No.: US 2025/0297983 A1
(43) Pub. Date: Sep. 25, 2025

(54) METHOD FOR MANUFACTURING PALM TRUNK-LIKE HIERARCHICAL NANOSTRUCTURE AND PALM TRUNK-LIKE HIERARCHICAL NANOSTRUCTURE MANUFACTURED THEREBY

(71) Applicant: Pukyong National University Industry-University Cooperation Foundation, Busan (KR)

(72) Inventors: Byeong-il LEE, Busan (KR); Rafiq AHMAD, Busan (KR)

(73) Assignee: Pukyong National University Industry-University Cooperation Foundation, Busan (KR)

(21) Appl. No.: 18/677,388

(22) Filed: May 29, 2024

(30) Foreign Application Priority Data
Mar. 19, 2024 (KR) 10-2024-0037929

(51) Int. Cl. G01N 27/327; C25D 1/00; C25D 1/20; C25D 5/48

(52) U.S. Cl. CPC G01N 27/3271; C25D 1/006; C25D 1/20; C25D 5/48; G01N 27/3276

(57) ABSTRACT

The present disclosure relates to a method for manufacturing a palm trunk-like hierarchical nanostructure and a palm trunk-like hierarchical nanostructure manufactured thereby. Specifically, the palm trunk-like hierarchical nanostructure according to the present disclosure demonstrates high specificity and sensitivity for glucose when glucose oxidase is immobilized on the surface thereof, with excellent reproducibility and stability, thus finding advantageous applications as biosensors.

### CROSS-REFERENCE TO RELATED APPLICATIONS

This application claims benefit of priority to Korean Patent Application No. 10-2024-0037929 filed Mar. 19, 2024, the entire content of which is incorporated herein by reference.

### BACKGROUND

### 1. Field of the Invention

The present disclosure relates to a method for manufacturing a palm trunk-like hierarchical nanostructure and a palm trunk-like hierarchical nanostructure manufactured thereby.

### 2. Description of the Prior Art

Quantitative or qualitative analysis of analytes present in biological samples is chemically or clinically important. For instance, this includes measuring the sugar level in the blood to manage the blood sugar level of diabetic patients, or measuring the level of cholesterol that causes adult diseases.

Currently, biosensors are mainly used for this purpose. A biosensor is a device capable of selectively detecting trace amounts of biological substances for analysis by combining a biological receptor that can recognize specific biological materials with an electrical or optical transducer that converts biological interactions and recognition responses into electrical or optical signals. In other words, it is used to measure glucose, uric acid, proteins, and DNA present in biological samples such as saliva or blood, requiring rapid and reproducible measurements.

Generally, a biosensor is composed of a sensor matrix consisting of a bioreceptor or biomimetic receptor that selectively recognizes the analyte, and a transducer that conveys signals generated during the reaction. Blood glucose biosensors, which are representative of biosensors using electrochemical methods, are broadly divided into the first and second generations. The first-generation blood glucose biosensors measure glucose levels by monitoring changes in the concentration of reduced oxygen or the hydrogen peroxide generated through the enzyme's redox reaction. Meanwhile, the second-generation blood glucose biosensors use a method where electrons generated through the enzyme's redox reaction are transferred to the electrode via an electron transfer mediator.

In this context, research on biosensors with high sensitivity and reproducibility is actively being conducted. In this regard, Korean Patent No. 10-2360124 relates to an electrochemical biosensor for measuring bio-signals, comprising carbon nanotubes and a manufacturing method therefor. This electrochemical sensor for continuous glucose measurement consists of an electrode on which a sensing membrane comprising redox enzymes, electron transfer mediators, and crosslinking substances is fixed along with carbon nanotubes. The sensor is disclosed as a continuous glucose measurement sensor that rapidly transfers electrons obtained from oxidizing the target substance by the enzyme through electron transfer mediators and carbon nanotubes.

### SUMMARY

The present disclosure aims to provide a method for manufacturing a palm trunk-like hierarchical nanostructure and a palm trunk-like hierarchical nanostructure manufactured using by the method.

Also, the present disclosure is to provide a biosensor including the palm trunk-like hierarchical nanostructure.

To achieve these aims, the present disclosure provides a method for manufacturing a palm trunk-like hierarchical nanostructure, the method including the steps of: sequentially depositing metal and zinc oxide onto a substrate; and heating the substrate coated with metal and zinc oxide in an aqueous solution of zinc nitrate hydrate and a heterocyclic compound to grow zinc oxide.

Additionally, the present disclosure provides a palm trunk-like hierarchical nanostructure manufactured by the manufacturing method.

Furthermore, the present disclosure provides a biosensor containing the palm trunk-like hierarchical nanostructure.

The palm trunk-like hierarchical nanostructure according to the present disclosure demonstrates high specificity and sensitivity for glucose when glucose oxidase is immobilized on the surface thereof, with excellent reproducibility and stability, thus finding advantageous applications as biosensors.

### BRIEF DESCRIPTION OF THE DRAWINGS

The above and other aspects, features and advantages of the present disclosure will be more apparent from the following detailed description taken in conjunction with the accompanying drawings, in which:

FIG. 1 is a schematic diagram illustrating the method for manufacturing a palm trunk-like hierarchical nanostructure-based sensor electrode according to the present disclosure;

FIGS. 2A-2D show FESEM images of the surface of the palm trunk-like hierarchical nanostructure, which were grown at 85° C. (FIG. 2A), 90° C. (FIG. 2B), 95° C. (FIG. 2C), or 100° C. (FIG. 2D) in one embodiment of the present disclosure;

FIGS. 3A-3D show FESEM images of the surface of the nanostructure formed on the sensor electrode fabricated in one embodiment of the present disclosure, observed at 25,000× (FIG. 3A), 50,000× (FIG. 3B), and 100,000× (FIG. 3C) magnifications and a TEM image of a cross-sectional image thereof (FIG. 3D);

FIGS. 4A-4C show images of the morphology of the nanostructure formed on the sensor electrode fabricated in one embodiment of the present disclosure, as observed by TEM (FIG. 4A) and HRTEM (FIG. 4B) and by SAED pattern analysis (FIG. 4C);

FIGS. 5A and 5B show an FESEM image (FIG. 5A) of the glucose oxidase immobilized to the sensor electrode fabricated in an embodiment of the present disclosure and a spectrum thereof as analyzed by EDX (FIG. 5B);

FIGS. 6A-6D show EIS plots (FIG. 6A), CV analysis curves measured at 50 mV/s (FIG. 6B) and 10-100 mV/s (FIG. 6C), and an oxidation-reduction peak plots (FIG. 6D) in the three-electrode cell setup including the sensor electrode fabricated in an embodiment of the present disclosure;

FIGS. 7A and 7B show curves illustrating the ability of the biosensor fabricated in one embodiment of the present disclosure to detect glucose in various pH conditions (FIG. 7A) and in the presence or absence of glucose (FIG. 7B);

FIGS. 8A-8C show amperometric responses according to glucose concentration (FIG. 8A), a calibration curve of current response vs. glucose concentration (FIG. 8B), and a calibration curve of 1/current response vs. 1/glucose concentration (FIG. 8C) in the biosensor fabricated in one embodiment of the present disclosure;

FIGS. 9A-9C show graphs confirming the selectivity (FIG. 9A), reproducibility (FIG. 9B), and durability (FIG. 9C) of the biosensor fabricated in one embodiment of the present disclosure; and

FIG. 10 is a plot confirming the ability of the biosensor fabricated in one embodiment of the present disclosure to sense the target substance in a clinical sample.

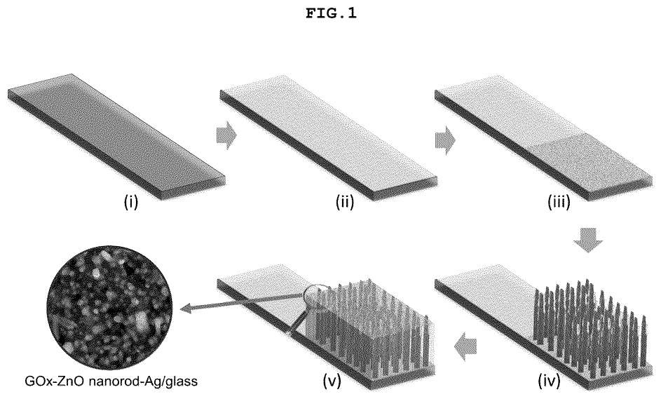

### DETAILED DESCRIPTION

Hereinafter, a detailed description will be given of the present disclosure.

The present disclosure provides a method for manufacturing a palm trunk-like hierarchical nanostructure, the method including the steps of: sequentially depositing metal and zinc oxide onto a substrate; and heating the substrate coated with metal and zinc oxide in an aqueous solution of zinc nitrate hydrate and a heterocyclic compound to grow zinc oxide.

The method for manufacturing a palm trunk-like hierarchical nanostructure according to the present disclosure includes a step of sequentially depositing metal and zinc oxide onto a substrate.

So long as it is known in the art, any substrate can be used. Specifically, the substrate may be prepared from at least one selected from the group consisting of glass, silicon (Si), acrylic, (PET), polystyrene, and polycarbonate, polyethylene terephthalate polypropylene. The substrate may be cleansed using conventional methods known in the relevant field before the deposition of metal and zinc oxide thereon.

The metal and zinc oxide deposited on the substrate may have a purity of 90% or higher, 93% or higher, 96% or higher, 97% or higher, 98% or higher, or 99% or higher. The deposition thickness of the metal and zinc oxide can be appropriately controlled by a person skilled in the art. For instance, metal may be deposited with a thickness of 300 nm or less, 250 nm or less, 200 nm or less, 150 nm or less, or 100 nm or less. Meanwhile, zinc oxide can be deposited with a thickness of to 500 nm, 10 to 400 nm, 10 to 300 nm, 10 to 200 nm, 10 to 100 nm, 10 to 70 nm, 30 to 500 nm, 30 to 400 nm, 30 to 300 nm, 30 to 200 nm, 30 to 100 nm, or 30 to 70 nm. Any metal may be deposited on the substrate as long as it is known in the art. For instance, the metal may be at least one selected from the group consisting of silver (Ag), gold (Au), chromium (Cr), platinum (Pt), copper (Cu), aluminum (Al), nickel (Ni), palladium (Pd), and titanium (Ti).

The deposition can be carried out using a method known in the art, and the method may be modified appropriately as necessary. Specifically, the deposition can be performed using physical vapor deposition (PVD), chemical vapor deposition (CVD), or atomic layer deposition (ALD). The term “physical vapor deposition” refers to a process where the material to be deposited is evaporated into gas form and condensed onto the relatively cooler substrate, leading to deposition. The physical vapor deposition may be conducted using deposition equipment like a thermal evaporator or E-beam evaporator, or through sputtering processes like magnetron sputtering or reactive sputtering. Meanwhile, the term “chemical vapor deposition” refers to formation of a material to be deposited on a substrate by using a gas containing the material to be deposited as a raw material. Chemical vapor deposition may include methods like thermal CVD, PECVD (plasma-enhanced CVD), HDPCVD (high-density plasma-enhanced CVD), PHCVD (photon-enhanced CVD), APCVD (atmospheric CVD), RPCVD (reduced pressure CVD), LPCVD (low pressure CVD), UHVCVD (ultra-high vacuum CVD), and MOCVD (metalorganic CVD). Furthermore, the term “atomic layer deposition” refers to a deposition method that builds atomic layers one by one.

The method for manufacturing a palm trunk-like hierarchical nanostructure according to the present disclosure includes a step of heating a substrate coated with metal and zinc oxide in an aqueous solution of zinc nitrate hydrate and a heterocyclic compound to grow zinc oxide.

The term “zinc nitrate hydrate”, as used herein, refers to a compound where water is bound to zinc nitrate, also called hydrated zinc nitrate. The hydrate may also contain water without a chemical bond to zinc nitrate. So long as it is known in the art, any zinc nitrate hydrate may be used. Examples of the zinc nitrate hydrate may be at least one selected from the group consisting of zinc nitrate dihydrate, zinc nitrate tetrahydrate, zinc nitrate hexahydrate, and zinc nitrate nonahydrate. In one embodiment of the present disclosure, the zinc nitrate hydrate is zinc nitrate hexahydrate.

In addition, the term “heterocyclic compound”, as used herein, refers to an organic compound with a ring structure that bears at least two atoms, such as carbon, nitrogen, oxygen, etc., as a ring member. So long as it is known in the art, any heterocyclic compound, whether synthetic or commercially available, may be used. By way of example, the heterocyclic compound may be at least one selected from the group consisting of hexamethylenetetramine (HMTA), triazine derivatives, melamine, and tetrazole. In one embodiment of the present disclosure, the heterocyclic compound may be hexamethylenetetramine.

The heating process may be conducted under a suitable condition to grow zinc oxide. Specifically, the heating may be carried out at a temperature of 80 to 100° C., 80 to 97° C., 83 to 100° C., 83 to 97° C., 86 to 100° C., 86 to 97° C., 89 to 100° C., 89 to 97° C., 92 to 100° C., or 92 to 97° C. Furthermore, the heating can be conducted for a duration of 1 to 50 hours, 1 to 45 hours, 1 to 40 hours, 1 to 35 hours, 1 to 30 hours, 1 to 25 hours, 3 to 50 hours, 3 to 45 hours, 3 to 40 hours, 3 to 35 hours, 3 to 30 hours, or 3 to 25 hours. Zinc oxide grown through the heating process can form a palm trunk-like hierarchical nanostructure.

The method may further include a step of immobilizing a bioreceptor after growing zinc oxide. The term “bioreceptor” refers to a substance that can detect a target by binding to or reacting chemically with the target. The method of immobilizing the bioreceptor on the nanostructure is well known in the field, and based on the type thereof, the bioreceptor can be immobilized to the nanostructure using appropriate methods and conditions. Specifically, the bioreceptor may include antibodies, aptamers, receptor proteins, enzymes, and the like. For instance, the bioreceptor may be glucose oxidase, cholesterol oxidase, uricase, urease, or peroxidase.

The nanostructure manufactured by the method according to the present disclosure, when glucose oxidase is immobilized thereto, can be applied to a biosensor for glucose detection. In another aspect, if cholesterol oxidase is immobilized on the nanostructure manufactured by the method according to the present disclosure, the nanostructure can be applied to a biosensor for detecting cholesterol. Similarly, if uricase is immobilized on the nanostructure manufactured by the method according to the present disclosure, the nanostructure can be applied in a biosensor for detecting uric acid. Furthermore, if urease is immobilized on the nanostructure manufactured by the method according to the present disclosure, the nanostructure can be applied for detecting urea in a biosensor. If peroxidase is immobilized on the nanostructure manufactured by the method according to the present disclosure, the nanostructure can be applied in a biosensor for detecting hydrogen peroxide.

The present disclosure also provides a palm trunk-like hierarchical nanostructure manufactured by the manufacturing method.

The palm trunk-like hierarchical nanostructure according to the present disclosure can be manufactured using the method described in the foregoing. Additionally, this nanostructure may have a bioreceptor immobilized thereto. If bioreceptors are immobilized thereto, the nanostructure can be used in a biosensor for detecting target substances, depending on the type of the targets to be detected by the bioreceptors.

Moreover, the present disclosure provides a biosensor containing the palm trunk-like hierarchical nanostructure.

The biosensor according to the present disclosure may include the hierarchical nanostructure manufactured using the method described in the foregoing. The target substance detected will depend on the type of bioreceptor immobilized to the nanostructure. For instance, if glucose oxidase is immobilized to the nanostructure, the biosensor can be applied for glucose detection. This detection may encompass determining the presence of glucose or quantifying the amount of glucose present.

A better understanding of the present disclosure may be obtained through the following Examples, which are set forth to illustrated, but are not to be construed to limit, the present disclosure.

### Example 1. Fabrication of Palm Trunk-Like Hierarchical Nanostructure-Based Sensor Electrode (1)

A palm trunk-like hierarchical nanostructure-based sensor electrode was fabricated as follows.

First, a glass slider with a size of 4×0.4 cm was sequentially cleansed with a detergent, water, acetone, and ethanal and then dried. A conductive silver (Ag) (purity 99.99%) thin film up to 100 nm thick was deposited on the cleansed glass slide using radio-frequency (RF) magnetron sputtering at a power of 60 W in an Ne atmosphere. In this regard, the operational conditions in the sputtering chamber were maintained at a pressure of 2.6×10⁻³ torr and room temperature. Then, zinc oxide with a high purity of 99.999% was used for RF sputtering in an argon (Ar) atmosphere at an RF powder of 60 W to deposit a zinc oxide seed layer with a size of 0.4×0.3 cm and a thickness of about 50 nm on the silver thin film-deposited glass slide. The sputtering chamber was maintained at a pressure of 6.5×10⁻³ torr and room temperature. Subsequently, the area of the silver thin film where the seed layer was not deposited was covered with a tape, and the glass slide was inverted and immersed in 100 ml of deionized water containing Zn(NO₃)₂·6H₂O (0.2 M) and hexamethylenetetramine (HMTA, 0.2 M) in a glass bottle. This glass bottle was placed in heating mantle and maintained at 90° C. for 20 hours to grow the zinc oxide. After completion of the reaction, the glass slide on which the zinc oxide nanostructure had grown was thoroughly rinsed to eliminate any impurities. Thus, a palm trunk-like hierarchical nanostructure-based sensor electrode was fabricated.

### Example 2. Fabrication of Palm Trunk-Like Hierarchical Nanostructure-Based Sensor Electrode-(2)

A palm trunk-like hierarchical nanostructure-based sensor electrode was fabricated in the same manner as in Example 1 with the exception of growing zinc oxide at 95° C. instead of 90° C.

### Comparative Example 1. Fabrication of Palm Trunk-Like Hierarchical Nanostructure-Based Sensor Electrode-(1)

A palm trunk-like hierarchical nanostructure-based sensor electrode was fabricated in the same manner as in Example 1 with the exception of growing zinc oxide at 85° C. instead of 90° C.

### Comparative Example 2. Fabrication of Palm Trunk-Like Hierarchical Nanostructure-Based Sensor Electrode-(2)

A palm trunk-like hierarchical nanostructure-based sensor electrode was fabricated in the same manner as in Example 1 with the exception of growing zinc oxide at 100° C. instead of 90° C.

### Example 3. Immobilization of Glucose Oxidase

Glucose oxidase (GO) was immobilized to the fabricated palm trunk-like hierarchical nanostructure-based sensor electrodes as follows.

In brief, the palm trunk-like zinc oxide hierarchical nanostructure formed on the sensor electrode fabricated in Example 2 was rinsed with PBS. The rinsed nanostructure was treated with 10 μl of PBS containing 10 mg/ml glucose oxidase and then reacted at 4° C. for 12 hours. After completion of the reaction, the nanostructure was washed and 5 μl of a thin nafion layer was applied to the surface of the nanostructure to afford a palm trunk-like hierarchical nanostructure-based sensor electrode with glucose oxidase immobilized thereto.

### Experimental Example 1. Nanostructure Formation According to Growth Temperature

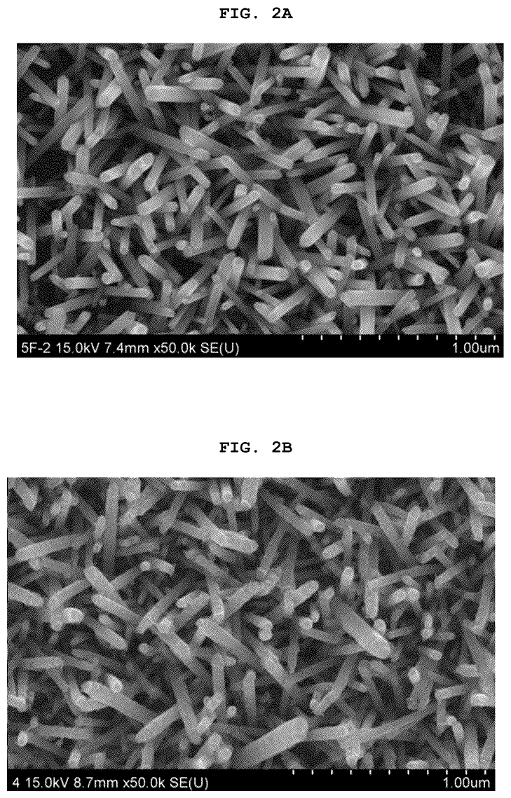

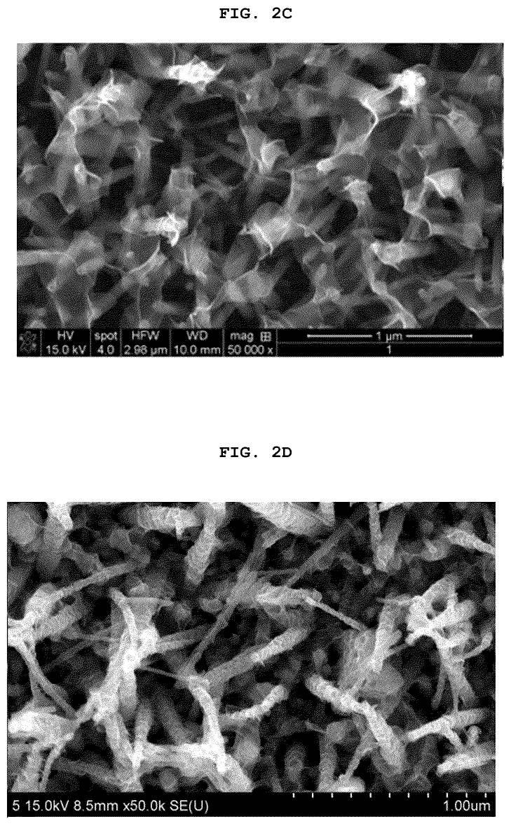

Using a field emission scanning electron microscope (FESEM, Hitachi S4700), observation was made of the surfaces of the nanostructures with zinc oxide grown at different temperatures, fabricated in Examples 1 and 2. The results are depicted in FIG. 2. In this regard, the nanostructures fabricated in Comparative Examples 1 and 2 were used as controls.

As shown in FIG. 2, the surfaces of the nanostructures fabricated in Examples 1 and 2 had palm trunk-like hierarchical nanostructure morphologies whereas the nanostructure fabricated in Comparative Example 1 had a smooth surface and the nanostructure fabricated in Comparative Example 2 did not exhibit consistent rod patterns.

### Experimental Example 2. Morphological Characterization of Nanostructure-(1)

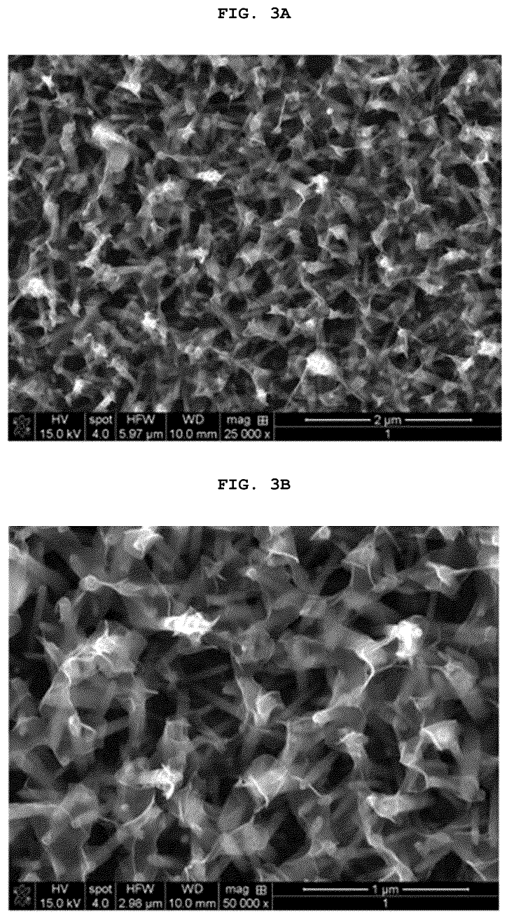

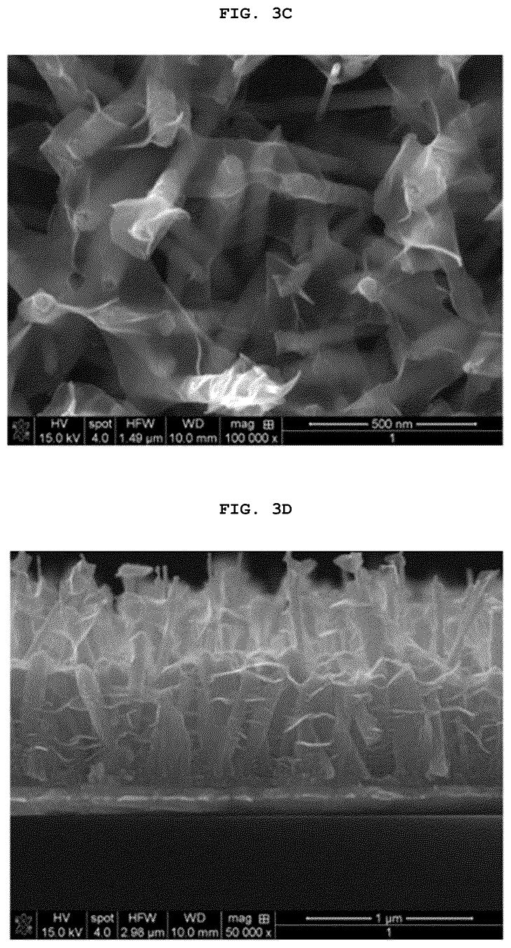

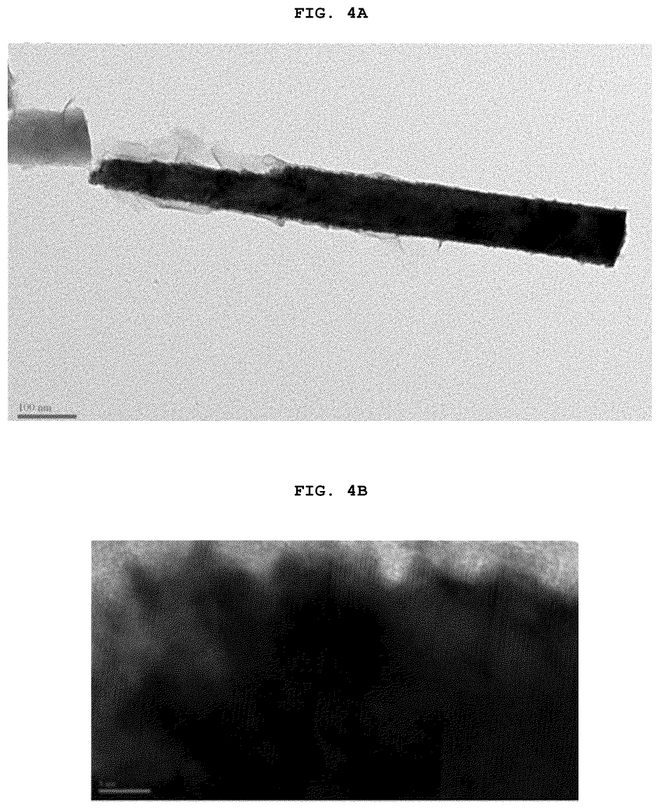

Using a field emission scanning electron microscope, observation was made of the surface of the zinc oxide nanostructure of the sensor electrode fabricated in Example 2. The results are depicted in FIGS. 3A to 3C. The cross section of the nanostructure was observed under a transmission electron microscope (TEM, JEOL-JEM-2010) equipped with a digital charge-coupled device (CCD), and the results are depicted in FIG. 3D.

As shown in FIGS. 3A to 3C, the zinc oxide nanostructure formed on the sensor electrode according to the present disclosure formed palm trunk-like hierarchical structure. Also, As shown in FIG. 3D, the zinc oxide nanostructure had a high degree of uniformity and were vertically oriented. The vertical length of the palm trunk-like zinc oxide hierarchical nanostructures ranged approximately between 1,400 to 1, 600 nm and were embellished with sheet-like nanostructures.

### Experimental Example 3. Morphological Characterization of Nanostructure-(2)

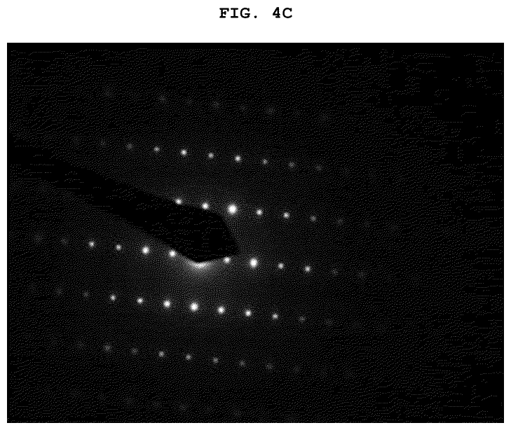

The surface morphology and crystallinity of the zinc oxide nanostructure of the sensor electrode fabricated in Example 2 was examined by TEM or high-resolution transmission electron microscopy (HRTEM). The results are depicted in FIGS. 4A and 4B. In addition, the nanostructure was analyzed for selected area electron diffraction (SAED) pattern and the analysis results are depicted in FIG. 4C.

The TEM image of FIG. 4A showed the zinc oxide nanostructure, characterized by a nanorod morphology with sheet-like structures adorning the surface of the ZnO nanorod. The HRTEM image of FIG. 4B distinctly showcases the crystal lattice of ZnO, aligning with the (002) plane (PCPDF #89-1397).

Meanwhile, as shown in FIG. 4C, the zinc oxide nanostructure had the ZnO's wurtzite crystal structure and confirmed the crystal lattice with the absence of any potential defects or impurities.

### Experimental Example 4. Immobilization of Glucose Oxidase

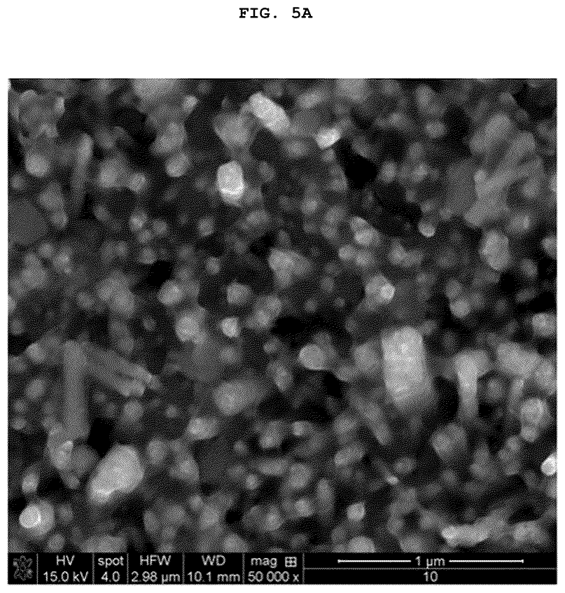

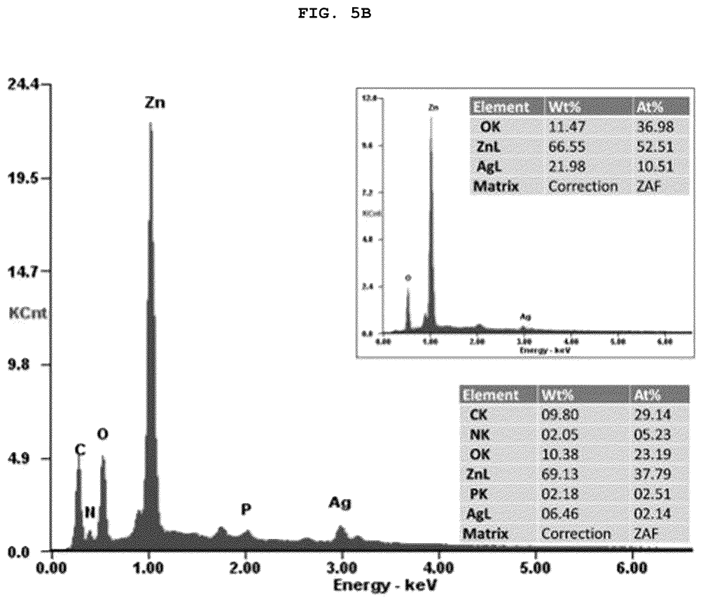

The immobilization of glucose oxidase on the zinc oxide nanostructure of the sensor electrode fabricated in Example 3 was examined using FESEM, and the results are depicted in FIG. 5A. In addition, elemental analysis of the nanostructure was performed by a typical method using an energy dispersive X-ray spectrometer (EDX). The result of elemental analysis is given in FIG. 5B.

As shown in FIG. 5A, the FESEM image clearly shows that glucose oxidase is uniformly distributed on the surface of the zinc oxide nanostructure. Furthermore, EDX analysis confirmed the presence of elements such as oxygen (O), zinc (Zn), carbon (C), nitrogen (N), and phosphorus (P) in the zinc oxide nanostructure.

### Experimental Example 5. Electrochemical Characterization of Sensor Electrode

The sensor electrode fabricated in Example 3 was examined for electrochemical properties as follows.

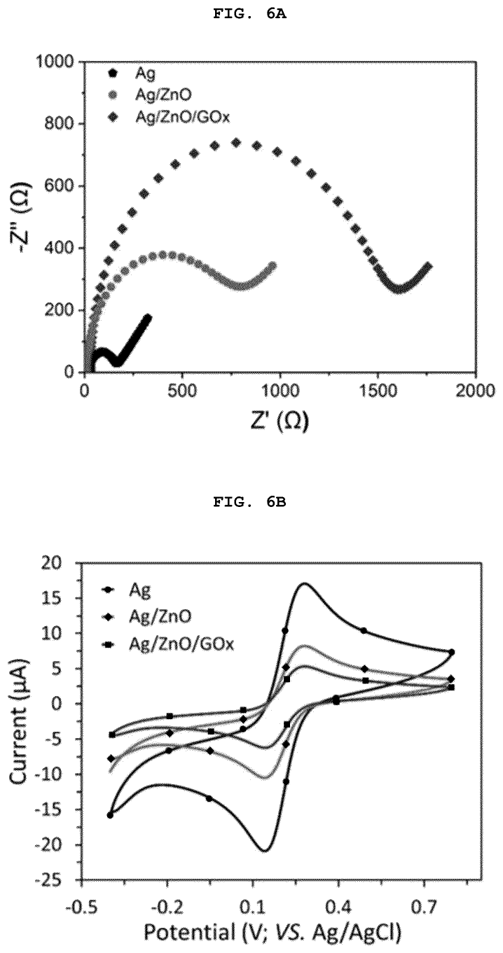

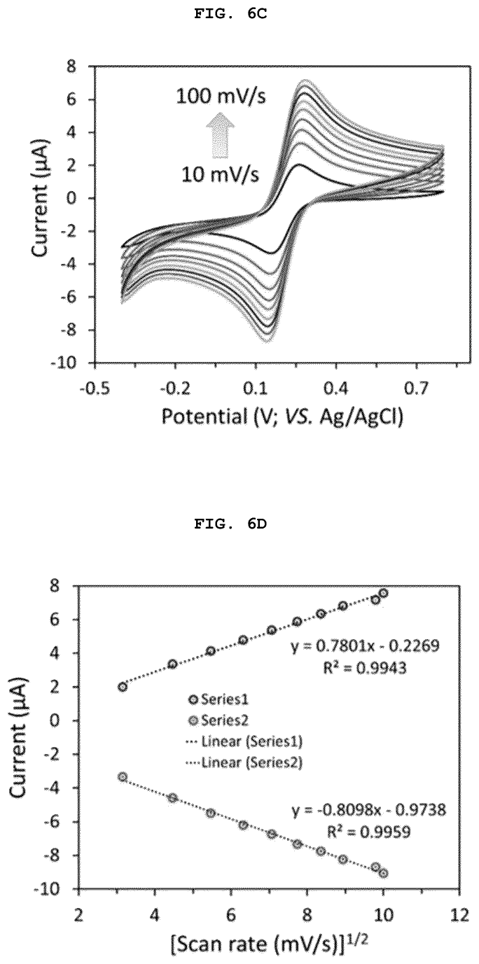

First, a typical three-electrode cell setup comprising the nanostructure fabricated in Example 3 as a working electrode, platinum wire as a counter electrode, and Ag/AgCl as a reference electrode was prepared. For a control, Ag or the nanostructure fabricated in Example 2 was employed in a three-electrode cell setup. Electrochemical impedance spectroscopy (EIS) was conducted on the cell setups, for which the measurements were performed with KCl (0.1 M) solution containing K₄Fe(CN)₆ and K₃Fe(CN)₆ (1:1 ratio; 5 mM each). The frequency range for the EIS measurements was spanned from 10⁻² to 10⁵ Hz with an applied potential of 0.30 V. The resulting measurements of charge-transfer resistance (Rct) are depicted in FIG. 6A. During the cyclic voltammetry (CV) and amperometric testing, the solution was consistently stirred at a speed of 100 rpm to ensure uniformity. The results are depicted in FIGS. 6B and 6C. Furthermore, oxidation-reduction peaks of the cell setups prepared were analyzed and the results are depicted in FIG. 6D.

As shown in FIG. 6A, the sensor electrodes according to the present disclosure increased in charge-transfer resistance with the decrease of electroconductivity due to the presence of glucose oxidase on the nanostructure surface. As shown in FIGS. 6B and 6C, the sensor electrode according to the present disclosure showed a lower current response compared to the control, and the current response increased with the increase of scan rates. In addition, as shown in FIG. 6D, the linear relationship of the oxidation-reduction current with the scan rate indicates that the sensor's response is predominantly governed by diffusion processes.

From the results, it is understood that the sensor electrodes fabricated into nanostructures according to the present disclosure work normally.

### Experimental Example 6. Glucose Detection by Sensor

### 6-1. Differential Pulse Voltammetry (DPV)

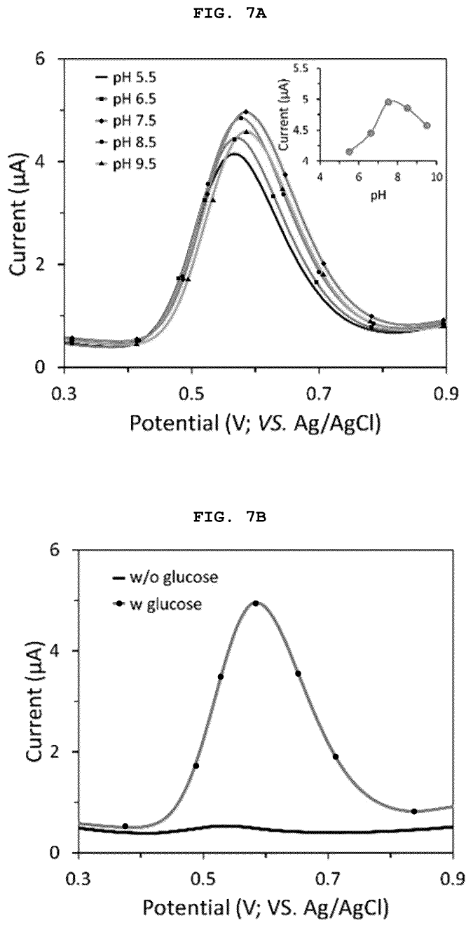

A biosensor was constructed in a typical method with the zinc oxide nanostructure fabricated in Example 3 and assayed for glucose sensing ability by DPV. Experiments were carried out in a typical manner using samples containing 0.2 mM glucose with a pH of 5.5 to 9.5. Samples with or without 0.2 mM glucose were subjected to DPV analysis. Analysis results from sample with or without 0.2 mM glucose at different pH values are depicted in FIG. 7.

As can be seen in FIG. 7A, the biosensor according to the present disclosure showed a maximum current response between pH 7 to 8, demonstrating an excellent detection effect in the conditions similar to the physiological condition (pH 7.4). Furthermore, as depicted in FIG. 7B, a well-defined oxidation peak of glucose was detected at +0.58 V only in the presence of glucose, indicating specificity for glucose.

### 6-2. Amperometric Method

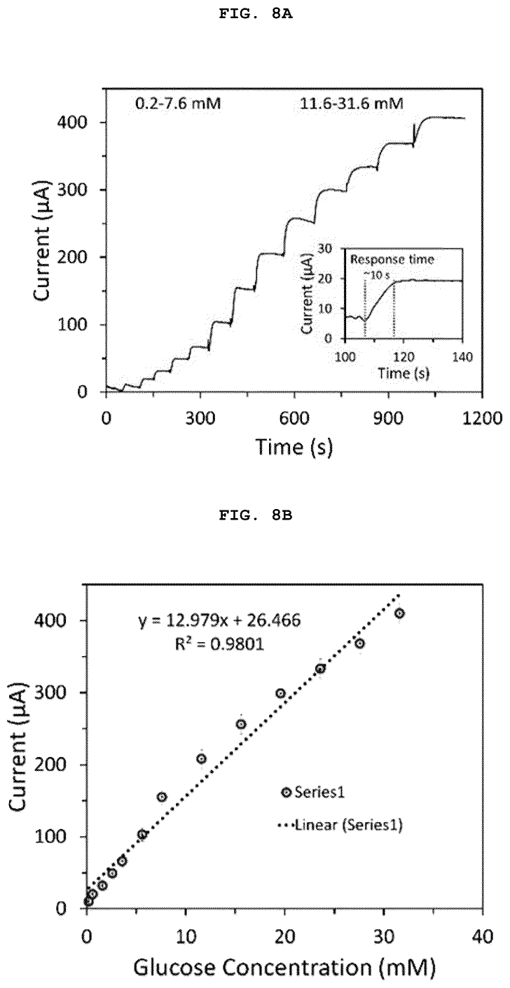

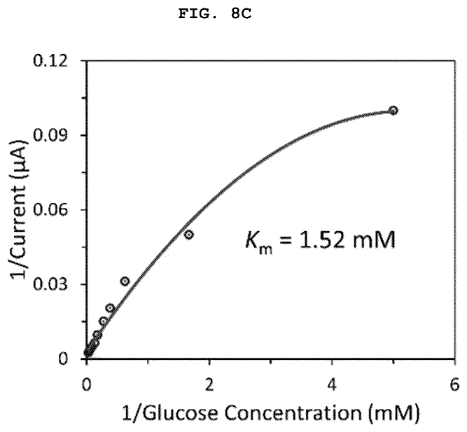

The sensing parameters including detection range, sensitivity, detection limit, and response time of the biosensor constructed with the fabricated zinc oxide nanostructure were measured using the amperometric method. Experiments were carried out with successive addition of 0.1 M PBS containing glucose at the applied potential of 0.58 V, and the results are given in FIG. 8A. For further evaluation, a linear calibration plot of current response vs. glucose concentration is presented in FIG. 8B and a calibrated plot of 1/current response vs. 1/glucose concentration is plotted in FIG. 8C.

As can be seen in FIG. 8A, the amperometric current response of the biosensor increased in a glucose concentration-dependent manner, with a maximum response attained in up to 10 seconds. From the plot of FIG. 8B, the biosensor according to the present disclosure detected glucose concentration linearly up to 31.6 mM, indicating the ability to cover a wide range of glucose concentrations. From the data plot of FIG. 8C, the Kₘ value of the biosensor is calculated to be 1.52 mM. This small Kₘ value indicates favourable enzyme-substrate kinetics and excellent affinity towards glucose.

### Experimental Example 7. Sensor Selectivity

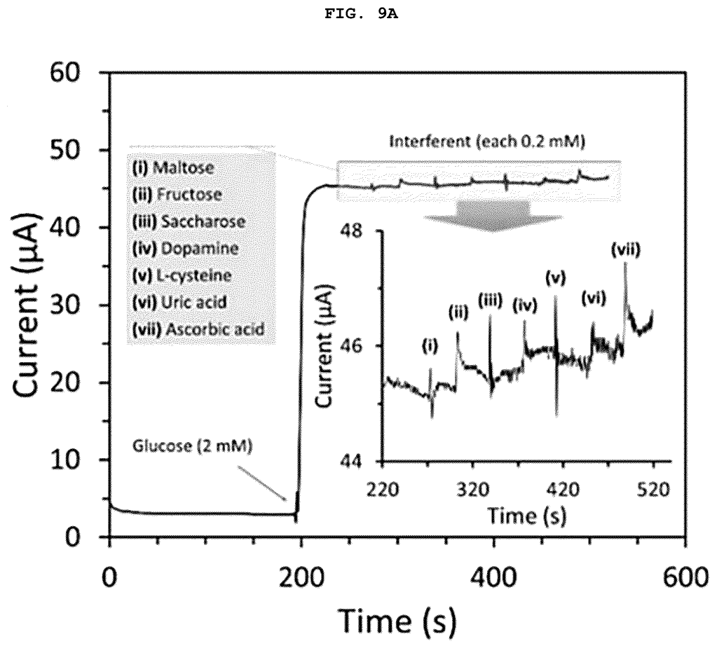

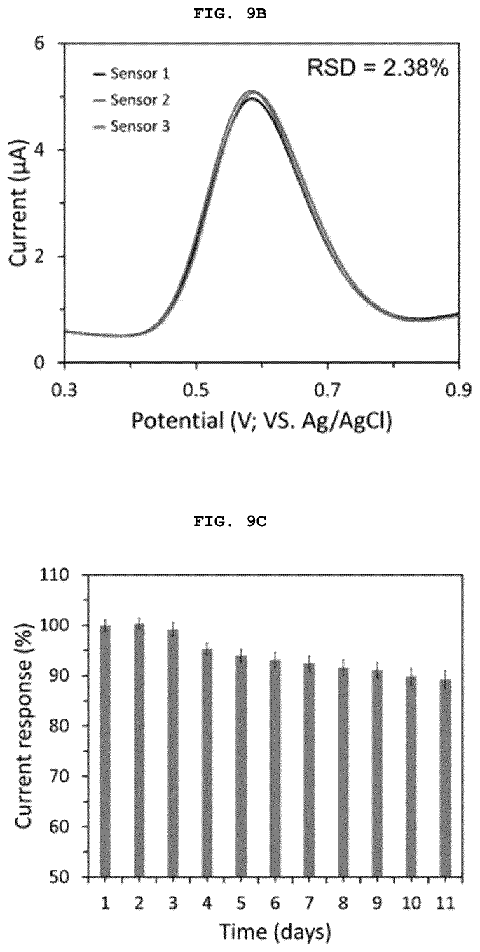

An amperometric method was employed to verify if the glucose sensing ability of the biosensor, constructed with the zinc oxide nanostructure from Example 3, was specific to glucose. The experiment followed the same procedure as in Experimental Example 6-2, except that a mixture containing 0.2 mM of glucose, maltose, saccharose, dopamine, L-cysteine, uric acid, and ascorbic acid was added sequentially. The current responses measured by the biosensor are presented in FIG. 9A. As shown in FIG. 9A, the biosensor represented consistent responses even when glucose was mixed with other substances, indicating that the sensor can accurately detect glucose without interference from other compounds.

### Experimental Example 8: Sensor Reproducibility

To verify the reproducibility of the biosensor, three zinc oxide nanostructures were fabricated using the method described in Example 3, and biosensors were made using same. The biosensors were tested for detecting 0.2 mM glucose using the DPV method, as outlined in Experimental Example 6-1, and the results are presented in FIG. 9B.

As shown in FIG. 9B, the relative standard deviation (RSD) was 2.38%, demonstrating high reproducibility across the three biosensors.

### Experimental Example 9: Sensor Durability

To verify the durability of the biosensor, glucose detection at 0.2 mM was conducted over 11 days using the DPV method, following the procedures described in Experimental Example 8. The results are presented in FIG. 9C.

As shown in FIG. 9C, the biosensor retained over 90% of its current response after 11 days, indicating stable glucose sensing ability.

### Experimental Example 10: Detection in Clinical Samples

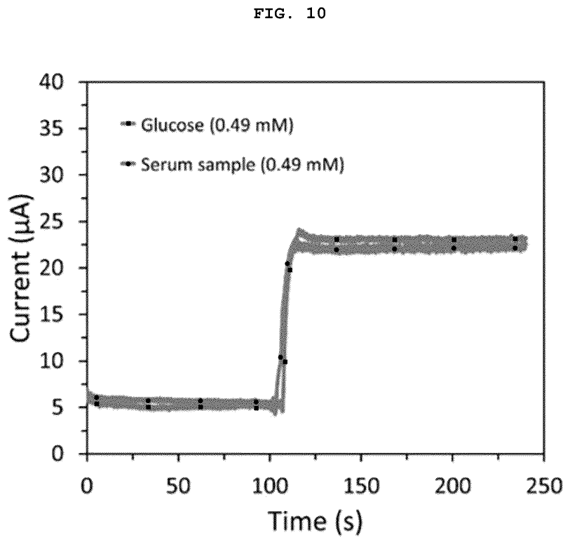

The glucose sensing ability of the biosensor according to the present disclosure was verified using serum samples in the following manner. The experiment was carried out in the same procedure as in Experimental Example 6-2, except that human serum containing 0.49 mM of glucose used as a sample. The results are presented in FIG. 10. As a control, PBS buffer containing the same concentration of glucose used.

As shown in FIG. 10, the biosensor according to the present disclosure demonstrated excellent glucose sensing ability even with clinical samples.

What is claimed is:

1. A method for manufacturing a palm trunk-like hierarchical nanostructure, the method comprising the steps of:
sequentially depositing metal and zinc oxide onto a substrate; and
heating the substrate coated with metal and zinc oxide in an aqueous solution of zinc nitrate hydrate and a heterocyclic compound to grow zinc oxide.

2. The method of claim 1, wherein the substrate is prepared from at least one selected from the group consisting of glass, silicon (Si), acrylic, polycarbonate, polyethylene terephthalate (PET), polystyrene, and polypropylene.

3. The method of claim 1, wherein the zinc nitrate hydrate is at least one selected from the group consisting of zinc nitrate dihydrate, zinc nitrate tetrahydrate, zinc nitrate hexahydrate, and zinc nitrate nonahydrate.

4. The method of claim 1, wherein the heterocyclic compound may be at least one selected from the group consisting of hexamethylenetetramine (HMTA), triazine derivatives, melamine, and tetrazole.

5. The method of claim 1, wherein the deposition may be performed using physical vapor deposition (PVD), chemical vapor deposition (CVD), or atomic layer deposition (ALD).

6. The method of claim 1, wherein the heating is carried out at 80 to 100° C.

7. The method of claim 1, wherein the heating is carried out for 1 to 50 hours.

8. The method of claim 1, further comprising a step of immobilizing a bioreceptor after the growth of zinc oxide.

9. A palm trunk-like hierarchical nanostructure, manufactured by the manufacturing method of claim 1.

10. A biosensor, comprising the palm trunk-like hierarchical nanostructure of claim 9.
